(19) United States
(12) Patent Application Publication
(10) Pub. No.: US 2004/0023363 A1
(43) Pub. Date: Feb. 5, 2004

(54) Biofiltering system for treating air

(76) Inventor: Bernhard Van Dyk, Burnaby (CA)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/381,920

(22) PCT Filed: Sep. 28, 2001
(86) PCT No.: PCT/CA01/01384

(30) Foreign Application Priority Data
Sep. 29, 2000 (CA) 2,321,404

(51) Int. Cl. A61L 9/01

(52) U.S. Cl. 435/266

(57) ABSTRACT

A filter system for removing organic, odor-causing substances from air has a filter bed which includes porous, particulate, non-compostable material and microorganisms capable of digesting the odor-causing substances. The particulate material of the bed is preferably lava rock, pumice or sandstone. A method of filtering air which contains organic, odor-causing substances, such as exhaust air from a process of thermophilic bacterial digestion of organic waste matter, includes providing a filter bed of particulate, porous, non-compostable material, inoculating the filter bed with a culture of microorganisms and passing the air through the filter bed.

### TECHNICAL FIELD

The invention pertains to an apparatus and method for filtering gases and, more specifically, to the filtering of air containing organic, odor-causing substances. The invention has particular application to filtering air exhausted from apparatuses for carrying out microbiological conversion of organic wastes, for example the thermophilic aerobic bacterial digestion of organic matter.

### BACKGROUND OF THE INVENTION

Processes for the thermophilic aerobic digestion of organic matter by microorganisms are well known in the art. In such processes, an aqueous slurry of the organic matter, for example waste vegetable matter, is brought into intimate contact with a suitable aerobic bacterial culture in a reaction vessel at appropriate conditions of temperature, pH and aeration. The bacteria digest the organic matter, producing simpler substances which may be used for fertilizer and other applications.

Such digestion processes, and the handing of the organic matter prior to digestion, produce odor-causing substances. For example, in some processes the organic waste material may be in a semi-decomposed state at the outset. The material may be mechanically macerated to a finely divided state prior to the digestion process itself. The microbiological digestion can also produce or release odor-causing substances from the organic matter. Within the digester, aeration is carried out which typically involves the injection of large volumes of air into the slurry, carrying odor-causing substances out of the slurry and into the airspace above the slurry in the digester.

The odor-filled air in the digesters, and in a plant where such digestion processes are carried out, must be exhausted from the digesters and plant. It is important to remove or reduce the odor-causing substances in the air before releasing it into the atmosphere, particularly where such processes are carried out in urban areas.

The odor-causing substances in such exhaust air are organic compounds and it is known that such compounds can be digested by microorganisms. In prior art filtering systems, the air to be filtered is typically passed through a bed of compostable material, such as wood or bark chips, having sufficient bacterial content to digest the odor-causing substances. However, in such prior art filter beds, the compostable material may calcify or harden, becoming substantially impermeable to to the air to be filtered. Further, being composed of organic material itself, the bed decomposes over time. Such filter beds require regular maintenance and replacement in order to function effectively.

### SUMMARY OF INVENTION

It is an object of the invention to provide a method and filter system for removing odor-causing substances from air in an effective manner, while requiring less filter bed maintenance and replacement than in the prior art systems.

The invention provides a filter system for removing organic, odor-causing substances from air, comprising a filter bed and a means for conducting the air into the filter bed. The filter bed comprises non-compostable, porous, particulate material, with microorganisms capable of digesting the odor-causing substances.

According to another aspect of the invention, the filter system includes a support for the filter bed which is porous to air and water, an air inlet for conducting air to be filtered to the filter bed and an outlet for exhausting filtered air to the atmosphere.

The invention also provides a method of filtering air which contains organic, odor-causing substances. A filter bed is provided which comprises particulate, porous, non-compostable material. The filter bed is inoculated with a culture of microorganisms capable of digesting the ordor-causing substances. The air to be filtered is then passed through the filter bed.

These and other objects, advantages and features of the invention will be apparent from the following description of the preferred embodiments, considered along with the accompanying drawings.

### BRIEF DESCRIPTION OF THE DRAWINGS

FIG. 1 is a schematic, vertical cross-sectional view through the longitudinal mid-line of a filter apparatus according to the invention.

FIG. 2 is a schematic cross-sectional view on the line 2-2 of FIG. 1.

### DESCRIPTION OF THE PREFERRED EMBODIMENTS

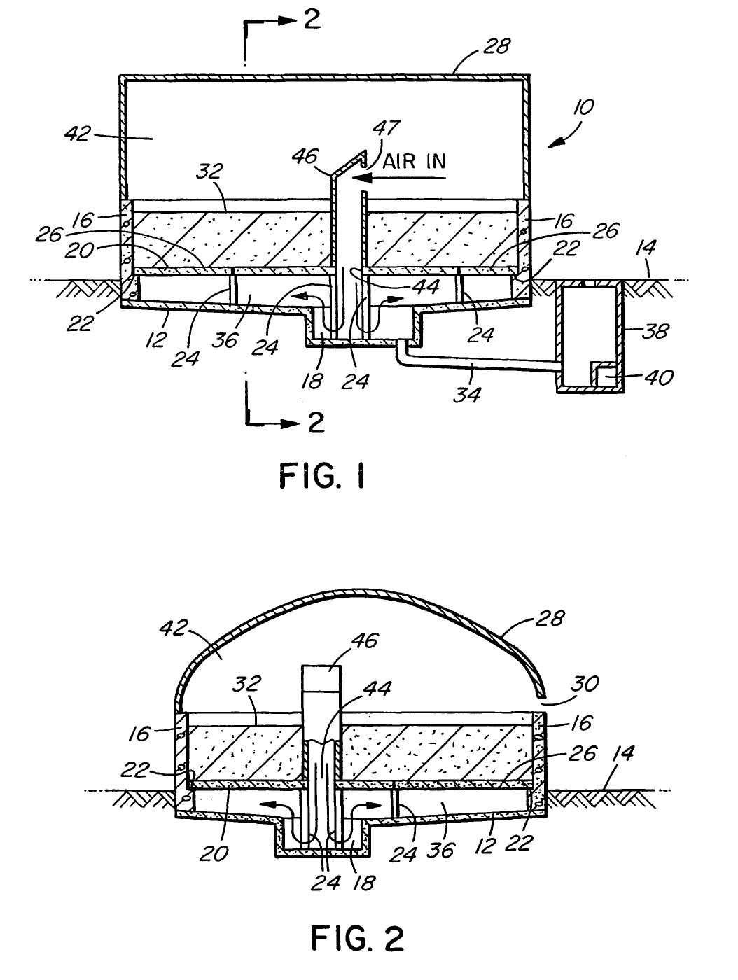

FIGS. 1 and 2 illustrate an air filter system embodying the principles of the invention. Filter apparatus 10 is preferably a freestanding structure constructed exterior to the digestion plant. It is rectangular in plan view and has a concrete base floor 12 constructed below ground level 14 and concrete wall 16 around the perimeter of the base floor. Base floor 12 slopes downward toward a central pit area 18 of the base floor for water drainage purposes as further described below.

Filter bed support floor 20 is spaced above the base floor 12, at approximately ground level. Support floor 20 is supported around its perimeter by ledges 22 on wall 16 and by posts 24 positioned on base floor 12. Support floor 20 comprises a number of individual rectangular sections 26 rather than being a single, integral unit. Sections 26 are made of reinforced concrete and have drainage slots therein (not shown in the drawings) to permit water to drain downward through the support floor and air to rise upward through it. Preferably, conventional cattle grate slabs are used as the rectangular sections 26 to make the support floor 20. Such slabs are ten feet long, four feet wide and four inches thick, with suitable drainage slots. Thus, for a support floor which is sixty feet long and thirty feet wide, forty-five slabs are used to construct to the floor. FIG. 1 illustrates only a few representative posts 24, and it is to be understood that a post is positioned at the corners of the slabs 26 (except where they are supported by ledges 22).

Roof 28 is constructed atop walls 16, covering the filter bed. Air venting space 30, shown in FIG. 2, is provided between one edge of roof 28 and one wall 16. Roof 28 keeps direct sunshine off the filter bed, preventing it from drying out, and also keeps rain from falling on the bed and comprising the controlled environment of the bed.

A screen of fibreglass netting (not shown in the drawings) of a mesh size commonly used for window screening, is laid directly on the top surface of support floor 20. The filter bed 32 is laid on top of this netting. The bed 32 is comprised of particulate, porous, non-compostable material. Preferably, the material is pumice or sandstone and, most preferably, lava rock. Such materials are highly porous and capable of absorbing a substantial amount of water. The particle size is preferably up to 2 inches in diameter and more preferable about ¾ inch to 1 inch in diameter. The filter bed is about four feet thick. The filter bed consists only, or substantially only, of the lava rock or other particulate, porous, non-compostable material and necessary microorganisms. Compostable material is neither necessary nor desirable in the filter bed.

Water drainage pipe 34 leads from the lowest point in the pit area 18 of airspace 36 between the base floor 12 and the support floor 20 into sump 38. A pump 40 in sump 38 is provided to pump waste water to a sewer or drainage filed.

In order to function effectively, the filter bed 32 must have within it copious amounts of bacteria capable of digesting odor-causing substances in the air to be filtered. This is preferably achieved by thoroughly moistening the filter bed so the porous material of the bed fills with water and inoculating the bed with the bacteria used in the digestion process itself, for example by spreading an aqueous solution of the bacterial culture on the top of the bed and allowing it to permeate the bed thoroughly.

Exhaust air to be filtered from the digestion plant and digesters is fed by an air duct and blower (not shown in the drawings) into exhaust air intake duct 46. Duct 46 is a square, box-like structure situated on support floor 20, surrounded by the filter bed and covering an opening 44 in support floor 20. In the preferred embodiment, duct 46 and opening 44 are approximately 4 feet in diameter. Exhaust air to be filtered is fed into the upper end of duct 46 through opening 47 therein and it passes through opening 44 into air space 36. It diffuses through air space 36 and rises up through the slots in support floor 20 into filter bed 32. There, the odor-causing substances come into contact with, and are digested by, the bacteria on and in the lava rock. A filter bed having an area of 30 feet by 60 feet and a depth of about 4 feet can effectively filter 15,000 cubic feet of air per minute.

Exhaust air coming from the digestion plant and the digesters is normally saturated with water. Much of this moisture condenses when the air contacts the filter bed 32. The condensate drains through the drainage holes in the support floor 20, onto base floor 12 and out through drain pipe 34 to sump 38.

The incoming air must be sufficiently moist in order for the bacterial digestion of the odor-causing substances to take place efficiently. In the event that relative humidity of the incoming air is less than about 65%, water is sprayed into the incoming air to moisten it prior to filtration.

The preferred embodiments described above are intended to illustrate the principles of the invention, but not to limit the scope of the invention. Various other embodiments and modifications to these preferred embodiments may be made by those skilled in the art without departing from the scope of the following claims.

What is claimed is:

1. A filter system for removing organic, odor-causing substances from air, comprising:
(a) a filter bed consisting essentially of non-compostable, porous, particulate material, said filter bed containing microorganisms capable of digesting said odor-causing substances; and
(b) means for conducting said air into said filter fed.

2. A filter system for removing organic, odor-causing substances from air, comprising:
(a) a filter bed consisting essentially of non-compostable, porous, particulate material, said filter bed containing microorganisms capable of digesting said odor-causing substances; and
(b) a support for said filter bed, said support being porous to air and water;
(c) an air inlet for conducting air to be filtered to said filter bed; and
(d) an air outlet for exhausting filtered air leaving said filter bed to the atmosphere.

3. A filter system according to claim 2, further comprising a roof to cover said filter bed.

4. A filter system according to claim 1, wherein said air comprises exhaust air from the thermophilic bacterial digestion of organic waste matter.

5. A filter system according to claim 1 or 2 wherein said particulate material is lava rock.

6. A filter system according to claim 1 or 2 wherein said particulate material is pumice.

7. A filter system according to claim 1 or 2 wherein said particulate material is sandstone.

8. A method of filtering air containing organic odor-causing substances, comprising the steps of:
(a) providing a filter bed consisting essentially of particulate, porous, non-compostable material;
(b) inoculating said filter bed with a culture of microorganisms capable of digesting said odor-causing substances; and
(c) passing said air through said filter bed.

9. A method according to claim 8 wherein said particulate material is lava rock.

10. A method according to claim 8 wherein said particulate material is pumice.

11. A method according to claim 8 wherein said particulate material is sandstone.
